(19) United States
(12) Patent Application Publication
(10) Pub. No.: US 2016/0166630 A1
(43) Pub. Date: Jun. 16, 2016

(54) A COMPOSITION HAVING A PREBIOTIC EFFECT

(71) Applicant: AMINO UP CHEMICAL CO., LTD., Kiyota, Sapporo (JP)

(72) Inventors: Sybille Buchwald-Werner, Düsseldorf (DE); Hajime Fujii, Ebetsu (JP); Tomohiro Ito, Sapporo (JP); Shotaro Kudo, Sapporo (JP)

(21) Appl. No.: 14/905,167

(22) PCT Filed: Jul. 18, 2014
(86) PCT No.: PCT/IB2014/001359

§ 371 Date: Jan. 14, 2016

(30) Foreign Application Priority Data
Jul. 18, 2013 (EP) 13 177 070.3

(51) Int. Cl. A61K 36/535

(52) U.S. Cl. CPC A61K 36/535; A23L 1/3002; A23L 1/308

(57) ABSTRACT

Provided are food, dietary supplements and/or drug compositions having prebiotic effect and their use in human and animal care.

### TECHNICAL FIELD OF THE INVENTION

The invention relates to a food, a dietary supplement, a functional food, a medical device and/or a drug composition which are characterized by comprising Perilla, and especially its prebiotic effect, to be used in human or animal care.

### BACKGROUND OF THE INVENTION

The microbial flora of the human intestine is a precisely balanced system within the digestive tract. More than 1000 different microbial species can be found. The dominant population consists of strictly anaerobe bacteria, like e.g. Bacteroides and Bifidobacterium. Subdominant flora includes bacteria belonging to the genera Streptococcus and Lactobacillus, and to a lesser extent Enterococcus, Clostridium and yeasts. Most of these species have a beneficial role, but others are potentially pathogenic, as some species of Clostridium, although the small number and competition with other bacteria prevent their proliferation and their pathogenic action. [1]

The gastrointestinal flora is responsible for different functions. Primary defence is one key function, preventing the colonization of pathogenic microorganisms and modulating the immune system by inducing the production of immunostimulants. In addition, the microflora plays an important role in regulating digestion.

The quality of the gastrointestinal flora depends on several parameters. Physiological parameters are age, stress and dietary intake. In addition, diseases, especially diarrhea, inflammatory bowel disease or irritable bowel syndrome, colitis and Crohn's disease as well as consumption of drugs, especially antibiotics, may alter the microflora negatively.

Consumption of prebiotics, probiotics and synbiotics has beneficial effects to stabilize and improve the gastrointestinal mircroflora. There is extensive literature that defines the beneficial health effects of consumption of prebiotic ingredients. Prebiotics may have beneficial effects to improve digestion, to prevent and/or reduce durations and complaints of gastrointestinal (GI) infections and to prevent and reduce inflammatory bowel disease (IBD) or irritable bowel syndrome (IBS) and/or colon cancer. In addition to beneficial effects to various gastrointestinal symptoms it has been shown that prebiotic agents have various other effects. In particular, prebiotics have beneficial effects to the immune system alleviating allergies and ameliorating immune diseases. They improve the mouth flora and prevent caries. In addition, prebiotics are known to support the reduction of inflammations, and atopic dermatitis. They improve mineral absorption and consequently improve bone density and stability and regulate lipid metabolism, lower cholesterol and triacylglycerol plasma concentration. Also beneficial effects for weight management by improved glucose homeostasis have been reported. [3, 4, 5]

A prebiotic is defined as a food ingredient that has the potential to improve host health by selectively stimulating the growth and/or activity of one or a limited number of bacteria in the colon. Firstly, beneficial microbial strains utilize the prebiotic substance and show improved growth or metabolize the prebiotic substance and decrease the pH value of the surrounding environment, thereby restricting or inhibiting the growth of non-beneficial/pathogenic microorganisms. In addition, the beneficial microbial strains have enhanced production of short chain fatty acids (SCFA), e.g. butyric acid, which has been shown to decrease the risk of bowel cancer. Furthermore, the growth of pathogenic bacteria is suppressed either directly by the prebiotic substance or indirectly by SCFA production of beneficial strains after metabolization of the prebiotic substance. Known prebiotics are dietary fibres such as inulin, soyfibers, lactulose, pectins, fructo-oligosaccharides (FOS), galacto-oligosaccharides (GOS), isomalto-oligosaccharides (IOS), xylo-oligosaccharides, glucosylsucrose (GS), lactusucrose (LS), palatinose-oligosaccharide (PAO), malto-oligosaccharide (MOS), gums and/or hydrolysates thereof, breast milk oligosaccharides, chitosan, which are able to survive the digestion and selectively stimulate the beneficial members of the gut microflora, such as Bifidobacteria, in the colon. [2, 3]

Despite considerable efforts by academic researchers and pharmaceutical and food industry, there is still a need for new prebiotic substances, to stabilize and improve the gastrointestinal microflora and consequently support and maintain good health and to improve and maintain gut health and to prevent and reduce gastrointestinal problems, or to treat gastrointestinal diseases. Existing prebiotics like fiber show side effects, like bloating and can be even contra productive for people suffering from inflammatory bowel conditions. In addition, the effective dosage of many prebiotics requires several grams, which are not easy to consume and lead to low consumer compliance. Therefore innovative prebiotic compositions with low, comfortable, effective dosages and without side effects, like bloating or gastrointestinal discomfort, are needed.

The object of the present invention was to provide novel ingredients to enhance the gastrointestinal microflora and to enhance consequently health and the gastrointestinal well-being, suitable as food, functional food, dietary supplement, medical device ingredients and/or drugs.

### SUMMARY OF THE INVENTION

The inventors surprisingly found that a composition comprising a Perilla preparation can be used as a prebiotic, preferably for promoting growth of probiotic bacteria. Thus, in one embodiment the invention relates to a composition comprising a Perilla preparation for use in promoting growth of probiotic bacteria in a subject as a prebiotic. Preferably, the preparation is a Perilla extract and more preferably a liquid, tea or powder. In particular, the inventors surprisingly showed that a composition comprising a Perilla preparation promotes or stimulates the growth of probiotic bacteria and/or neutralizes (inhibits) pathogenic bacteria, in particular reduces or inhibits growth of the pathogenic bacteria.

Surprisingly, the inventors have shown that the Perilla preparation promotes the growth of probiotic bacteria but not of pathogenic bacteria such as Enterobacteriaceae, Bacteroides and/or Clostridia. Therefore, in one embodiment, the invention relates to a Perilla preparation for use as a prebiotic for use in promoting growth of probiotic bacteria in a subject, wherein the preparation is capable of (i) promoting the growth of probiotic bacteria (e.g. Bifidobacteria and/or Lactobacilli); and/or (ii) inhibiting the growth of pathogenic bacteria (e.g. Enterobacteriaceae, Bacteroides and/or Clostridia) or having a neutral effect on growth of pathogenic bacteria (e.g. Enterobacteriaceae, Bacteroides and/or Clostridia).

Furthermore, the invention relates to a composition comprising Perilla, preferably comprising an additional probiotic ingredient, which allows a synergistic effect. In particular, the invention further relates to a synbiotic agent comprising a composition comprising Perilla and a probiotic agent.

In a preferred embodiment, the Perilla preparation is a Perilla frutescens or Perilla ocymoides preparation, more preferably Perilla frutescens (L.) Britton var. Frutescens, Perilla frutescens var. crispa, Perilla frutescens var. purpurascens, or Perilla frutescens var. hirtella.

Preferably, the preparation is a preparation of aerial parts of Perilla, preferably a leaf or stem preparation, more preferably a fresh or dried leaf preparation.

Furthermore, the preparation is preferably an extract comprising vicenin 2, preferably the extract is standardized.

Surprisingly, it could be shown that the Perilla preparation promotes growth of probiotic bacteria, preferably selected from the group consisting of Bifidobacteria and Lactobacilli, more preferably, selected from the group consisting of Lactobacillus bulcaricus, Lactobacillus plantarum, Lactobacillus acidophilus, Lactobacillus fermentum, Lactobacillus casei, Lactobacillus rhamnosus, Bididobacterium lactis, Bifidobacterium bifidum and Bifidobacterium longum. The most preferred strains in this respect are Lactobacillus bulcaricus, Lactobacillus acidophilus, Bifidobacterium bifidum and Bifidobacterium longum. Promoting growth of probiotic bacteria improves the gastrointestinal microflora and consequently supports the gut health and/or prevents or reduces gastrointestinal problems, e.g. reducing infectious diseases of gut.

In addition, either independently or in combination to the characteristic that Perilla preparation promotes growth of probiotic bacteria, the inventors surprisingly found that Perilla extract can be used for neutralizing pathogenic bacteria. The neutralizing of pathogenic bacteria may occur, e.g. preferably by reducing or inhibiting, i.e. delaying or preventing, the growth of pathogenic bacteria. The pathogenic bacteria can, for example, be selected from the group consisting of Enterobacteriaceae, Bacteroides and Clostridia, preferably, selected from the group consisting of Salmonella typhimurium, Escherichia coli and Enterobacter cloacae. It is to be emphasized that neutralization or reducing the growth of pathogenic bacteria in gut consequently reduces infectious diseases of gut.

In a further preferred embodiment the composition comprises a further prebiotic agent (such as a fiber), a probiotic agent, a lipid, a physiologically active fatty acid, a sterol, a sterol ester, a stanol ester, a bulking agent, a medicament, an antispasmodic or anti-inflammatory agent, plant phenolic, a phenolic metabolite, a mineral, a vitamin, an essential oil or a plant preparation. In this context, it is to be emphasized that in the composition the Perilla preparation has prebiotic activity, i.e. is the active ingredient.

In one embodiment, the Perilla preparation is a Perilla plant preparation capable of (i) promoting the growth of Bifidobacteria and Lactobacilli; and (ii) inhibiting the growth of Enterobacteriaceae, Bacteroides and/or Clostridia.

Preferably, the further prebiotic agent is a fiber (e.g. kiwi fiber, baobab fiber or soyfiber), an inuline, pectin, lactulose, oligosaccharide types, fructo-oligosaccharides (FOS), galacto-oligosaccharides (GOS), isomalto-oligosaccharides (IOS), xylo-oligosaccharides, glucosylsucrose (GS), lactusucrose (LS), palatinose-oligosaccharide (PAO), malto-oligosaccharide (MOS), arabinogalactan, chitosan and chitosan derivates, psyllium, gums and/or hydrolysates thereof or breast milk oligosaccharides.

Preferably, the further probiotic agent is a Bifidobacteria, Lactobacilli, a yeast or mixtures thereof.

In an additional preferred embodiment, the plant phenolic is selected from a group consisting of anthocyanins, procyanidins, flavonoids, flavanones, flavonols, catechins, tannins and isoflavones. The essential oils, preferably, include mint oil or kiwi fruit oil.

Preferably, the medicament is a further prebiotic agent, an antibiotic, an anti-infective agent, a tricyclic antidepressant, a cholecystokinin-1 antagonist, a serotonergic agent, a benzodiazepine or analogue, a neurokinin antagonist, a Guanylate cyclase-C agonist, a C1-C2 channel activator, a C1 secretion blocker, a GLP1 analogue, a κ-opioid agonist, an antacid, a sodium phosphate, NA reuptake inhibitor, omeprazol analogue, glycocorticoid, statine, anti-histaminica, vitamin B complex (vitamin B 2,3,6,12), zinc, urea, cholesterin resorption inhibitor or cynara preparation.

Preferably, the plant preparation as a further agent is selected from one or more extracts from a group consisting of an extract of Aloysia triphylla, Hypericum perforatum, Hyperzia serrata, Galanthus nivalis, Salvia officinalis, Panex ginseng, Lippia citriodora, Melissa officinalis, Passiflora incarnate, Passiflora edulis, Bacopa monnieri, Zingiber officinalis, Leucojum aestrum, Concolulus pluricaulis, Centella asiatica, Emblica officinalis, Coptidis Rhizoma, Salvia triloba, Piper nigrum, Trigonella foenum-graecum, Cimicifuga racemosa, Salvia miltiorrhiza, Rhodiola rosea, Habranthus jamesonii, Phycella herbertiana, Rhodophiala mendocina, Zephyranthes filifolia, Stephania pierrei, Kaempfera parviflora, Stephania venosa, Crocus sativus, Salvia species, Bacopa monnieri, Centella asiatica, Ptychopetalum olacoides, Withania somnifera, Maytenus ilicifolia, Mentha spicata, Coriandrum sativum, Urtica Circularis, Lychnophora pohlii, Lychnophora trypanocidal, Jodina rhombifolio, Aspalathus linearis, Cyclopia species, Adansonia digitata, Sclerocarya birrea, Mangifera indica, Actinidia chinensis, Matricaria recutita, Mentha piperita, Plantago lanceolata, Morinda morindoides, Vitis viniferis, Psidium guajava, Aloysia citriodora, Cordyceps sinensis, Laminaria digitata, Cynara scolymus, Carum carvi, Iberis amara, Angelica archangelica, Silybum marianum, Chelidonium majus, Glycyrrhiza glabra, Vaccinium macrocarpon, Vaccinium myrtillus, Vaccinium corymbosum, Vaccinium ashei, Vaccinium angustifolium, Prunus virginiana, Vaccinium parvifolium, Gaylussacia species, Vaccinium vitis-idaea, Amelanchier alnifolia Nutt, Cynara species, Salix species or combinations thereof. In a more preferred embodiment, the plant preparation is selected from Aloysia triphylla, Lippia citriodora, Melissa officinalis, Piper nigrum, Mentha spicata, Aspalathus linearis, Cyclopia species, Adansonia digitata, Mentha piperita, Aloysia citriodora, Vaccinium myrtillus, Zea mays, Lamiaceae, Verbenaceae, Scrophulariaceae or combinations thereof.

In a preferred embodiment, the concentration of the fresh Perilla leaves is from 2 mg to 400 g, preferably 400 mg to 40 g, more preferably 1000 mg to 10 g, 2000 mg to 6000 mg or about 4000 mg, the concentration of the dried Perilla leaves is from 0,25 mg to 50 g, preferably 50 mg to 5000 mg, more preferably 125 mg to 1250 mg, 250 mg to 750 mg or about 500 mg or the concentration of the Perilla preparation (extract or tea) is from 0.05 mg to 10 g, preferably 10 mg to 1000 mg, more preferably 25 mg to 250 mg, 5 mg to 150 mg or 10 mg to 100 mg.

As shown in the Examples Perilla preparation is effective as prebiotic and thus, no other prebiotic agent is necessarily needed. Therefore, in one embodiment the composition does not comprise other prebiotic and/or probiotic ingredients. Thus, in one embodiment, the composition consists of a Perilla preparation optionally with excipients. Thus, in one further embodiment the composition does not include further prebiotic and/or probiotic agents, e.g as stated in WO 2013/079623 which is incorporated herein. In particular, the composition according to the invention may be substantially free of further (i.e. other) prebiotic agents and/or probiotic agents.

The composition according to any one of preceding embodiments may be comprised in a food product, functional food product, beverage, dietary supplement, medical device or medicament. Put in other words the invention relates to a food product, functional food product, beverage, dietary supplement, medical device or medicament comprising the composition as defined above.

Furthermore, the preparation comprising Perilla may be used for maintaining digestive health, maintaining healthy gut microflora, preventing and reducing the duration and/or complaints of GI infections, like Enterobacteria infections, maintaining a normal digestion, improving gut regularity, supporting healthy gut mobility, bowel movement and/or healthy stool frequency, stool consistency and/or form, reducing bloating, reducing distension, reducing passage of gas, reducing stomach rumbling, reducing feeling of fullness, improving bowel function, ameliorating constipation, ameliorating diarrhea or reducing visceral hypersensitivity and/or abdominal discomfort or e.g. pain and cramps. In addition, the preparation comprising Perilla may be used to prevent and/or reduce inflammatory bowel disease or irritable bowel syndrome and/or colon cancer.

In addition, the preparation comprising Perilla may be used for improving the immune system, skin and/or hair, preventing skin and/or hair disorders, alleviating allergies, ameliorating immune diseases, preventing and/or reducing atopic dermatitis, regulating the lipid metabolism, lowering cholesterol and/or triacylglycerol plasma concentrations, improving glucose homeostasis and/or inducing beneficial effects for weight management. In this context, improving or inducing beneficial effects can be used interchangeably.

In addition, the composition may be used for use in prevention and/or reducing duration and complaints of gut infections, preferably, wherein the gut infection is food infection, including travel diarrhea. Moreover, the composition may be used for improving the mouth flora and/or preventing caries.

Put in other words, the invention relates to a composition comprising a Perilla preparation for use in promoting growth of probiotic bacteria in a subject and/or for inhibiting the growth of pathogenic bacteria selected from the group consisting of Enterobacteriaceae, Bacteroides and/or Clostridia in a subject for treating the above-mentioned conditions.

Thus, the invention relates to treating or ameliorating of diseases or conditions, wherein a prebiotic agent has a beneficial effect to a disease or condition in a subject.

In this regard, the person skilled in the art understands that a prebiotic agent has advantageous effects to the above-mentioned conditions. In addition, as mentioned above, promoting growth of probiotic bacteria improves the gastrointestinal microflora and consequently supports the gut health and/or prevents or reduces gastrointestinal problems. It is to be emphasized that neutralization or reduction of pathogenic bacteria in gut consequently reduces infectious diseases of gut.

Gastrointestinal infections are a major cause of morbidity and mortality worldwide. Studies conducted in 2006 found that, globally, severe diarrhea and dehydration are responsible each year for the death of 1,575,000 children under the age of five. This represents 15% of the 10.5 million deaths per year of children in this age group. The use of probiotics to prevent and treat a wide variety of conditions has gained favor in the past decade due to a need to find alternatives to traditional therapies such as antibiotics as well as the lack of good treatments for GI ailments. Identification of mechanisms by which probiotics may impact human health have been summarized in (Britton and Versalovic 2008).

Probiotics may provide an important strategy for the prevention and treatment of gastrointestinal infections. Probiotics may stimulate the host's immune function and mucosal barrier integrity. By working via different mechanisms of probiosis, probiotics may yield effects at different steps in the process. Probiotics may prevent disease from occurring when administered prophylactically. Probiotics may also suppress or diminish severity or duration of disease in the context of treatment.

The normal intestinal microbiota prevents the colonization of pathogenic bacteria and has important immune functions. Modem life style and eating habits contributed to a change in intestinal microflora lined to the development of diseases. Notwithstanding heterogeneity of population, results show that probiotics have beneficial effects against various diseases, particularly against infections, e.g. viral infections. Probiotics do not show antiviral effects by direct action on the virus, but by mechanism stimulating the immune system infections can be reduced (Iqbal et. al. 2014).

However, the invention does not relate to treating gastric ulcer or Helicobacter pylori infection.

The present invention further relates to a kit comprising the composition or preparation of any one of the preceding embodiments. In particular, the invention relates to the composition as defined above packaged in a kit.

The present invention provides a method of promoting growth of probiotic bacteria and/or inhibiting or reducing the growth of pathogenic bacteria by administering to an individual the preparation or composition comprising a Perilla preparation.

In addition, the invention relates to an in vitro use of composition comprising Perilla for promoting growth of probiotic bacteria and/or inhibiting or reducing the growth of pathogenic bacteria.

In one embodiment the invention relates to a method of inhibiting the growth of pathogenic bacteria selected from the group consisting of Enterobacteriaceae, Bacteroides and/or Clostridia in a subject comprising administering a Perilla composition as defined above or to a method of treating or preventing gut infections in a subject comprising administering a Perilla composition as defined above.

Preferably, the gut infection is travel diarrhea or food infection.

In addition, the invention relates to a use of composition as defined above, for promoting growth of probiotic bacteria in vitro or to a use of composition as defined above for inhibiting the growth of pathogenic bacteria, preferably selected from the group consisting of Enterobacteriaceae, Bacteroides and/or Clostridia, in vitro.

### BRIEF DESCRIPTION OF THE FIGURES

FIG. 1: Spotting scheme of probiotic strains for the preparation of growth experiments on agar plates.

FIG. 2: Growth of probiotic Lactobacillus strains on sMRS medium after 24 h of cultivation supplemented with plant extract formulation in comparison with glucose and without C-source. 1—Perilla extract with medium sMRS+1% C; 2—positive control with medium sMRS+1% glucose; 3—negative control with medium sMRS w/o C-source. Dilutions: 10⁻¹, 10⁻², 10⁻³, 10⁻⁴ (from left to right).

FIG. 3: Growth of probiotic Lactobacillus strains on LMM medium after 24 h of cultivation supplemented with plant extract formulation in comparison with glucose and without C-source. 1—Perilla extract with medium sMRS+1% C; 2—positive control with medium sMRS+1% glucose; 3—negative control with medium sMRS w/o C-source. Dilutions: 10⁻¹, 10⁻², 10⁻³, 10⁻⁴ (from left to right).

FIG. 4: Growth of probiotic Lactobacillus strains on sMRS medium after 48 h of cultivation supplemented with plant extract formulation in comparison with glucose and without C-source. 1—Perilla extract with medium sMRS+1% C; 2—positive control with medium sMRS+1% glucose; 3—negative control with medium sMRS w/o C-source. Dilutions: 10⁻¹, 10⁻², 10⁻³, 10⁻⁴ (from left to right).

FIG. 5: Growth of probiotic Lactobacillus strains on LMM medium after 48 h cultivation supplemented with plant extract formulation in comparison with glucose and without C-source. 1—Perilla extract with medium sMRS+1% C; 2—positive control with medium sMRS+1% glucose; 3—negative control with medium sMRS w/o C-source. Dilutions: 10⁻¹, 10⁻², 10⁻³, 10⁻⁴ (from left to right).

FIG. 6: Growth of probiotic bifidobacteria strains on special bifidobacterium medium after 48 h of cultivation supplemented with plant extract formulation in comparison with glucose and without C-source. 1—Perilla extract with medium sMRS+1% C; 2—positive control with medium sMRS+1% glucose; 3—negative control with medium sMRS w/o C-source. Dilutions: 10⁻¹, 10⁻², 10⁻³, 10⁻⁴ (from left to right).

FIG. 7: Growth of probiotic bifidobacteria strains on LMM medium after 48 h of cultivation supplemented with plant extract formulation in comparison with glucose and without C-source. 1—Perilla extract with medium sMRS+1% C; 2—positive control with medium sMRS+1% glucose; 3—negative control with medium sMRS w/o C-source. Dilutions: 10⁻¹, 10⁻², 10⁻³, 10⁻⁴ (from left to right).

FIG. 8: Growth of strain 9 (bifidobacterium longum subsp. longum) on bifidobacterium and LMM medium supplemented with plant extract formulation in comparison with glucose and without C-source after 96 h of cultivation. 1—Perilla extract with medium sMRS+1% C; 2—positive control with medium sMRS+1% glucose; 3—negative control with medium sMRS w/o C-source. Dilutions: 10⁻¹, 10⁻², 10⁻³, 10⁻⁴ (from left to right).

FIG. 9: Spotting scheme of non probiotic strains for the evaluation of growth on agar plates.

FIG. 10: Growth of enterobacteria on CASO medium supplemented with plant extract formulation in comparison with glucose and without C-source after 16 h of cultivation. 1—Perilla extract with medium sMRS+1 C; 2—positive control with medium sMRS+1% glucose; 3—negative control with medium sMRS w/o C-source. Dilutions: 10⁻¹, 10⁻², 10⁻³, 10⁻⁴ (from left to right).

FIG. 11: Growth of enterobacteria on CASO medium supplemented with plant extract formulation in comparison with glucose and without C-source after 24 h of cultivation. 1—Perilla extract with medium sMRS+1% C; 2—positive control with medium sMRS+1% glucose; 3—negative control with medium sMRS w/o C-source. Dilutions: 10⁻¹, 10⁻², 10⁻³, 10⁻⁴ (from left to right).

FIG. 12: Growth of Clostridia strains on M110 medium supplemented with plant extract formulation in comparison with glucose and without C-source after 24 h of cultivation. 1—Perilla extract with medium sMRS+1% C; 2—positive control with medium sMRS+1% glucose; 3—negative control with medium sMRS w/o C-source. Dilutions: 10⁻¹, 10⁻², 10⁻³, 10⁻⁴ (from left to right).

FIG. 13: Growth of Clostridia strains on M110 medium supplemented with plant extract formulation in comparison with glucose and without C-source after 48 h of cultivation. 1—Perilla extract with medium sMRS+1% C; 2—positive control with medium sMRS+1% glucose; 3—negative control with medium sMRS w/o C-source. Dilutions: 10¹, 10⁻², 10⁻³, 10⁻⁴ (from left to right).

FIG. 14: Growth of Bacteroides fragilis on M110 medium supplemented with plant extract formulation in comparison with glucose and without C-source after 7 days. 1—Perilla extract with medium sMRS+1% C; 2—positive control with medium sMRS+1% glucose; 3—negative control with medium sMRS w/o C-source. Dilutions: 10⁻¹, 10⁻², 10⁻³, 10⁻⁴ (from left to right).

### DETAILED DESCRIPTION OF THE INVENTION

The inventors surprisingly found that a composition comprising a Perilla preparation can be used as a prebiotic. Advantageously, a prebiotic composition comprising Perilla requires a low dosage of Perilla, and does not induce side effects. In addition, the effect included by Perilla arises within 24 h.

The present invention demonstrates for the first time that Perilla plant extract powder exhibits prebiotic properties as all of the selected probiotic strains showed growth on media supplemented with the Perilla plant extract powder as the only C-source. It is remarkable that some of these strains exhibited a growth on the Perilla plant extract comparable to growth on glucose, which is usually considered as the optimal C-source for microorganisms. The Perilla plant extract seems to have an outstanding growth promoting activity on probiotic microorganisms (in particular strain 2—Lactobacillus acidophilus, strain 7—Lactobacillus bulgaricus, strain 8—Bifidobacterium bifidum and strain 9 Bifidobacterium longum). This means that these strains exclusively used the native Perilla extract as the only C-source on the corresponding plates. These four strains are very suitable as a synbiotic (plant extract in combination with one these strains) with a Perilla preparation.

A major basic characteristic of a prebiotic substance is the ability to selectively stimulate or promote growth of probiotic bacteria (e.g., Bifidobacteria and Lactobacillii) while having no or even detrimental effects on growth of potentially pathogenic bacteria (e.g., Bacteroides, Clostridia) (6, 7). The results of the present invention demonstrate this crucial characteristic for the plant extract in vitro within single cultivation experiments.

However, this finding provides uses for gastrointestinal indications and, in addition, for several other indications relating to bacterial growth (e.g. maintaining healthy mouth flora and reducing caries), to immunological indications or to indications concerning mineral absorption, lipid metabolism, glucose metabolism and/or inflammatory processes.

Concerning the effect of the Perilla plant extract on the tested non-probiotic bacteria the present data show that the tested strains (facultative anaerobic and three obligate anaerobic bacteria) were not able to metabolize the Perilla plant extract as a C-source.

The growth of three pathogens, Salmonella typhimurium, Escherichia coli and Enterobacter cloacae was even inhibited (significantly delayed or completely inhibited) by the Perilla extract formulation (C).

All this underlines the prebiotic effect of the Perilla plant extract and indicates that the Perilla plant extract has the potential to induce a shift of the intestinal microflora towards beneficial and commensal bacterial species, which is the key property a prebiotic substance should exhibit. This is useful in treating gastrointestinal infections, caries or mouth infections.

The prebiotic properties of the Perilla plant extract powder during the gastrointestinal passage are also stable when applying the Perilla plant extract as a prebiotic dietary compound.

The novel prebiotic agent or preparation may be comprised i.a. in globules, pellets, powder formulation, tablets, capsules, stick formulation, sachet formulation or a fluid. The fluid may be in a bottle with a dropper. Besides oral application in capsules, tablets, also powder and granulate formulation being presented as ready to mix blend in a stick package are used. This stick formulation can be directly solved in water.

Perilla preparations and extracts thereof, in particular compositions comprising vicenin 2 are described in detail in international application WO 2013/079623 at page 8, line 17 to page 10, line 21, page 14, line 27 to page 15, line 12 and page 15, lines 15 to 26 as well as international application WO 2013/079624 at page 6, line 20 to page 9, line 6, page 15, line 3 to 18 and page 15, line 21 to page 16, line 3, the disclosure content which is incorporated herein by reference.

### Definitions

It is to be noted that the term “a” or “an” entity refers to one or more of that entity; for example, “preparation”, is understood to represent one or more preparations. As such, the terms “a” (or “an”), “one or more”, and “at least one” can be used interchangeably herein.

By “subject” or “individual” or “animal” or “patient” or “mammal”, is meant any subject, particularly a mammalian subject, e.g., a human patient, for whom diagnosis, prognosis, prevention, or therapy is desired.

As used herein, the term “prebiotic” refers to a substance consumed orally by an individual to beneficially affect that individual by selectively stimulating the growth and/or activity of one or more of a limited number of bacteria in the colon of the individual. The preferred prebiotics are those which selectively stimulate the growth and/or activity of Bifidobacteria and/or lactic acid bacteria (Lactobacilli). Even more preferred are those which stimulate the growth and/or activity of Bifidobacteria and/or lactic acid bacteria (Lactobacilli) in preference to pathogenic bacteria such as Enterobacteriaceae, Bacteroides or Clostridia.

The term “tea” refers to material from the aerial parts of Perilla extracted with water,

The term “aerial preparation” refers to parts of Perilla above the ground, i.e. leaves and stem.

“Further prebiotic agent” refers to dietary fibres such as inulin, soyfibers, lactulose, fructo-oligosaccharides, galacto-oligosaccharides, breast milk oligosaccharides, chitosan, which are able to survive the digestion and selectively stimulate the beneficial members of the gut microflora, such as Bifidobacteria, in the colon. Most prebiotic agents include non-starch polysaccharides and oligosaccharides poorly digested by human enzymes.

“Probiotic bacteria” according to this invention are beneficial bacteria found in the intestinal tract of healthy mammals, e.g. Bifidobacteria and Lactobacillus.

“Synbiotic agent” relates to an agent comprising a prebiotic agent and a probiotic agent in combination.

“Pathogenic bacteria” are bacteria that produce illness or disease and are harmful to the organism, e.g. Enterobacteriaceae, Bacteroides and Clostridia.

Having a “neutral effect to growth of pathogenic bacteria” relates to the growth of pathogenic bacteria without an accelerating or inhibiting effect.

The term “neutralizing pathogenic bacteria” refers to counteracting pathogenic bacteria or abolishing pathogenic effects of the bacteria, in other words inhibiting pathogenic bacteria. Neutralizing may occur via inhibiting the growth or reducing (preventing or delaying) the growth of pathogenic bacteria including bringing the growth back to a normal level.

One or more of the following testing methods which are known to a person skilled in the art for evaluating the prebiotic effect may be used. 1) In vitro Method: Fermentation of single microbial strain on agar plates by feeding with the prebiotic and counting of the bacterial growth. 2) In vitro Method: Fermentation of complex mixtures of bacteria on agar plates by feeding with the prebiotic and counting the growth of the different bacteria genera. 3) Acid-resistant testing- Treating of the prebiotic with gastric acid to mimic gastrointestinal influences and consequently apply the prebiotics to test 1) or 2) to proof if activity is still measurable. 4) Demonstrating prebiotic effects in vivo by feeding the prebiotic to animals or human and to analyze the bacterial composition in feces samples pre and post application of the prebiotic. (Roberfroid 2007)

“A functional food product” according to this invention is understood to be a food, beverage or infant formula product, which offers, in addition, to nutritional value a health benefit, which supports and improves health and wellbeing or helps to reduce the risk to develop a disease.

“A dietary supplement product” according to this invention are food products in form of a pill, tablet, capsule pellet, globule, powder or liquid form, which are meant to be taken by mouth, and contain substances like vitamins, minerals, foods, botanicals, amino acids and are intended to supplement the usual intake of these substances via the normal diet.

“A medicament/drug/medicine” according to this invention is any substance with the potential to prevent or cure disease or enhance physical or mental welfare. If not stated otherwise the term “drug”, “medicine”, or “medicament” are used interchangeably herein and shall include but are not limited to all (A) articles, medicines and preparations for internal or external use, and any substance or mixture of substances intended to be used for diagnosis, cure, mitigation, treatment, or prevention of disease of either man or other animals; and (B) articles, medicines and preparations (other than food) intended to affect the structure or any function of the body of man or other animals; and (C) articles intended for use as a component of any article specified in clause (A) and (B). The term “drug”, “medicine” or “medicament” shall include the complete formula of the preparation intended for use in either man or other animals containing one or more “agents”, “ingredients”, “compounds”, “substances” or “(chemical) compositions” as and in some other context also other pharmaceutically inactive excipients as fillers, disintegrants, lubricants, glidants, binders or ensuring easy transport, disintegration, disaggregation, dissolution and biological availability of the “drug”, “medicine”, or “medicament” at an intended target location within the body of man or other animals, e.g., at the skin, in the stomach or the intestine. The terms “agent”, “compound” or “substance” are used interchangeably herein and shall include, in a more particular context, but are not limited to all pharmacologically active agents, i.e. agents that induce a desired biological or pharmacological effect or are investigated or tested for the capability of inducing such a possible pharmacological effect by the methods of the present invention.

As used herein, the terms “treat” or “treatment” refer to both therapeutic treatment and prophylactic or preventative measures, wherein the object is to prevent or slow down (lessen) an undesired physiological change or disorder, such as the development of gastrointestinal infection. Beneficial or desired clinical results include, but are not limited to, alleviation of symptoms, diminishment of extent of disease, stabilization (i.e., not worsening) state of disease, delay or slowing of disease progression, amelioration or palliation of the disease state, and remission (whether partial or total), whether detectable or undetectable. Those in need of treatment include those already with the condition or disorder as well as those prone to have the condition or disorder or those in which the manifestation of the condition or disorder is to be prevented. In this context, improving, ameliorating or treating can be used interchangeably herein.

Unless stated otherwise, the terms “disorder” and “disease” are used interchangeably herein.

“Maintaining a healthy gut” according to the invention can be understood as maintaining a normal digestion with normal gut mobility and stool frequency, without pain and/or bloating.

“Constipation” as used herein can be defined as infrequent bowel movements, reduced bowel movement frequency as well as hard to pass bowel movements.

The term “healthy gut microflora” relates to a stable and balanced gut microflora, wherein the probiotic bacteria prevail the pathogenic bacteria so that no uncontrolled growth of pathogenic bacteria occurs and the individual has no complications in gut health.

“Traveler's disease or traveler's diarrhea” relates to a diarrhea of sudden onset, often accompanied by abdominal cramps, vomiting, and fever, occurring sporadically in travelers usually during the first week of the trip; most commonly caused by unfamiliar strains of pathogenic bacteria like E. coli species.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above. Moreover, the term “comprises” is meant to encompass the terms “consisting essentially of” and “consisting of”.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” or “Pubmed” may be utilized, which is hosted by the National Center for Biotechnology Information and/or the National Library of Medicine at the National Institutes of Health. Further databases and web addresses, such as the virtual library “Martindale's center” are known to the person skilled in the art and can also be obtained using internet search engines.

Several documents are cited throughout the text of this specification. The contents of all cited references (including literature references, issued patents, published patent applications as cited throughout this application and manufacturer's specifications, instructions, etc.) are hereby expressly incorporated by reference; however, there is no admission that any document cited is indeed prior art as to the present invention.

The above disclosure generally describes the present invention. A more complete understanding can be obtained by reference to the following specific examples which are provided herein for purposes of illustration only and are not intended to limit the scope of the invention.

### EXAMPLES

### Background and Aim of the Project:

Prebiotic potential of selected substances within in-vitro studies were studied by evaluating the utilization of the Perilla extract (formulation C) by probiotic microorganisms and further selected strains of Bifidobacterium and Lactobacillus as well as evaluating the effects of the plant extract on the growth of typical commensal and pathogenic species of the gastrointestinal tract.

### Example 1

### Perilla Promotes Growth of Probiotic Bacteria

### Pre-Selection of Strains

Table 1 summarizes the commercial probiotic strains which were tested in the examples.

TABLE 1
List of probiotic strains tested.

| Strain | Source |
|---|---|
| 1. Lactobacillus plantarum 299v | DSM strain no. 9843 |
| 2. Lactobacillus acidophilus | DSMZ strain no. 20079 |
| 3. Bificobacterium lactis | Bb-12 Chr Hansen |
| 4. Bifidobacterium longum subsp. infantis | DSMZ strain no. 20088 |
| 5. Lactobacillus fermentum | DSMZ strain no. 20391 |
| 6. Lactobacillus casei | DSMZ strain no. 20011 |
| 7. Lactobacillus bulgaricus | DSMZ strain no. 20081 |
| 8. Bifidobacterium bifidum | DSMZ strain no. 20456 |
| 9. Bifidobacterium longum subsp. longum | DSMZ strain no. 20219 |
| 10. Lactobacillus rhamnosus | DSMZ strain no. 20021 |

In addition to the probiotic strains, eight commensals or pathogenic strains were selected and also tested in this example.

Growth Experiments with Probiotic Strains on Agar Plates:

### Analysis of Strain 1-10

The growth behaviour of ten probiotic strains selected was tested on agar plates containing two different media compositions supplemented with different C (carbon)-sources.

The following cultivation trials were carried out on agar plates for each of the ten strains:
1. 2 cultivations per strain with Perilla extract as C-source (1%, weight per volume)—formulation C
2. 2 cultivations per strain with glucose as C-source (1%, weight per volume)—positive control 1
3. 2 cultivations per strain without glucose—negative control

The plant extract formulation was not additionally sterilized before using in the assay.

As media agar bases sMRS medium (synthetic MRS; MRS=de Man-Rogosa-Sharpe medium=typical medium for lactobacilli) and LMM medium (lactobacillus minimal medium) were used for growth experiments with lactobacilli. These two media were chosen, because sMRS represents a complex medium containing undefined compounds like yeast extract and peptone while LMM medium is a medium containing exclusively defined ingredients. For the growth experiments with Bifidobacteria, a special Bifidobacterium medium was chosen as complex medium and LMM medium supplemented with cysteine (0.5 g/l) was used as a defined medium.

The compositions of the media used are listed in tables 2, 3 and 4.

TABLE 2
Composition of sMRS medium.

| Substance | Manufacturer | Net weight | |
|---|---|---|---|
| Proteose peptone No. 3 | BD | 10 | g/l |
| Beef extract (BBL) | BD | 10 | g/l |
| Yeast extract | Biospringer | 5 | g/l |
| Diammoniahydrogene-citrate | Merck | 2 | g/l |
| ((NH₄)₂H-Citrate) | | | |
| Tween 80 | Merck | 1 | g/l |
| Magnesiumsulfate (MgSO₄ × 7 H₂O) | Merck | 206 | mg/l |
| Manganesesulfate (MnSO₄ × H₂O) | Sigma | 56 | mg/l |
| di-potassiumhydrogenephosphate | Merck | 2 | g/l |
| (K₂HPO₄) | | | |
| D(+)-glucose × H₂O | Merck | 20 | g/l |

TABLE 3
Composition of LMM medium.

| Substance | Manufacturer | Net weight | |
|---|---|---|---|
| di-Potassium hyrogene phosphate | Merck | 2 | g/l |
| di-Ammonium hydrogene citrate | Merck | 2 | g/l |
| Calcium chloride dihydrate | Sigma | 0.5 | g/l |
| Magnesiumsulfate heptahydrate | Merck | 0.6 | g/l |
| Guanine | Fluka | 0.1 | g/l |
| Cytosine | Fluka | 0.1 | g/l |
| Thymidine | Fluka | 0.1 | g/l |
| 2′-Desoxyuridine | Sigma | 0.1 | g/l |
| 2′-Desoxyadenosine | Sigma | 0.1 | g/l |
| Cyanocobalamine | Sigma | 0.02 | g/l |
| Riboflavin | Fluka | 10 | mg/l |
| Folic acid | Sigma | 0.2 | g/l |
| Pyridoxal-5-phosphate monohydrate | Aldrich | 10 | mg/l |
| Aminobenzoat | Fluka | 0.2 | g/l |
| D (+)-Biotin | Merck | 1 | mg/l |
| Ascorbic acid | Fluka | 500 | mg/l |
| Nicotinacid | Fluka | 10 | mg/l |
| Calciumpanthotenat | Sigma | 10 | mg/l |
| Thiamine | Fluka | 1 | mg/l |
| Cobalt-(II)-nitrat hexahydrate | Sigma | 0.5 | g/l |
| Manganese (II) sulfate | Sigma | 0.02 | g/l |
| Na₂MoO₄ | Fluka | 0.04 | g/l |
| Trypticase Peptone | Fluka | 15 | g/l |
| Tween 80 | Merck | 1 | g/l |
| D-Glucose | Merck | 20 | g/l |

TABLE 4
Composition of Bifidobacterium medium.

| Substance | Manufacturer | Net weight | |
|---|---|---|---|
| Proteose Peptone No. 3 | BD | 10 | g/l |
| Beef Extract (BBL) | BD | 5 | g/l |
| Yeast Extract | Biospringer | 5 | g/l |
| di-potassiumhydrogenephosphate | Merck | 3 | g/l |
| (K₂HPO₄) | | | |
| Sodium ascorbate | Roth | 1 | g/l |
| L-cysteine | Merck | 0.5 | g/l |

### Establishment of Experimental Set-Up and Preparation of Media

For the preparation of the cultivation media 20% (w/v) solutions (in water) of glucose and carbon source formulations were prepared. The Perilla extract formulation (C) was applied as non sterilized solutions. The 20% (w/v) solution of glucose was treated by sterilization.

The agar bases were supplemented with different carbon sources just before pouring the media into the petri dishes. The applied concentration of the sample formulation in each agar prepared was 1% (w/v). One positive control was prepared with 1% glucose. As negative control medium without addition of a C-source was prepared. In total three different preparations per medium were used.

The initial pH of the media was adjusted to a neutral pH value between 6 and 7 to simulate intestinal conditions. To determine whether the Perilla extract formulation has an influence on the pH value of the media, the pH value was also determined after addition of Perilla extract solution. This was done, since it is known that acidic supplements can cause a decrease of the pH value of a medium. Low pH values can cause an unwanted growth inhibition of bacteria, thereby making it difficult to compare the growth of bacteria in media with different supplements.

As an example, table 5 represents the results of pH measurements of sMRS medium after addition of plant extract formulation or glucose.

TABLE 5
pH values of sMRS medium containing the plant
extract formulation and glucose as a control.

| | Medium no | | |
|---|---|---|---|
| | 1 | 2 | 3 |
| Description | sMRS + 1% | sMRS + 1% | sMRS |
| | formulation | glucose | without C- |
| | C | | source |
| pH value | 6.43 | 6.62 | 6.64 |

The data in table 5 reveal that addition of plant extract formulation as well as glucose had no influence on the pH values of the tested media.

For the evaluation of the growth behaviour of the probiotic strains listed in table 1, the cells were pre-cultivated in MRS- or Bifidobacterium medium and cultivated at 37° C. for 24 h. Afterwards, the cells were harvested and resuspended in PBS to avoid possible influences of residual nutrients in the media on subsequent growth experiments on agar plates.

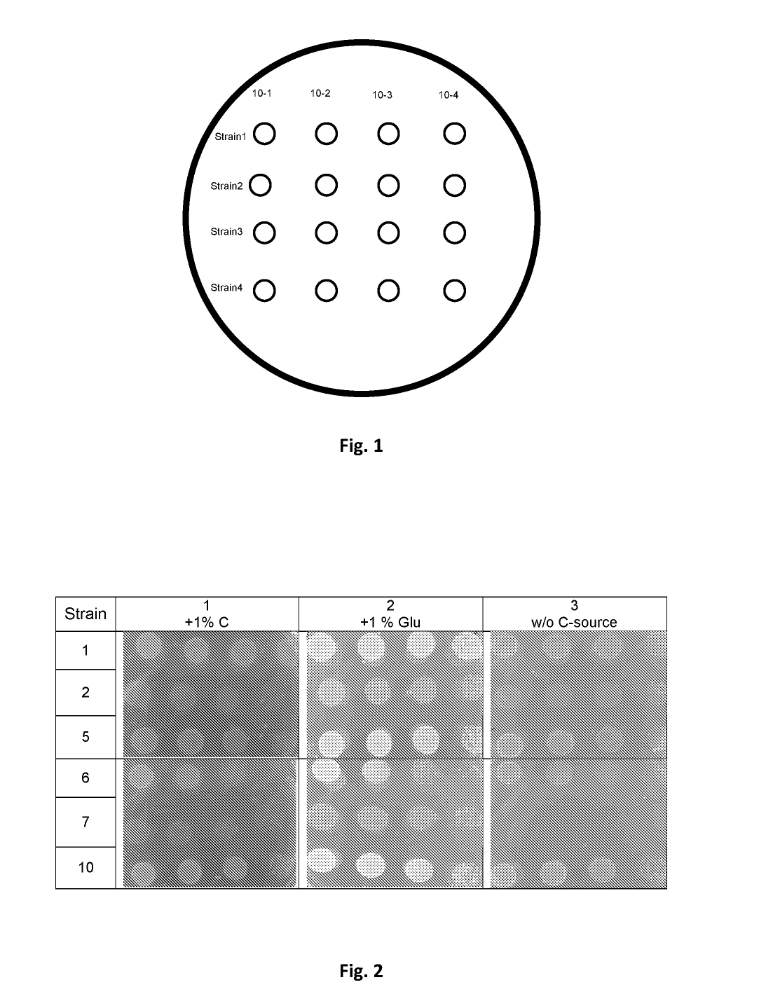

For the spotting on agar plates different dilutions of each of the probiotic strains (10⁻¹ to 10⁻⁴) were prepared. FIG. 1 shows the corresponding spotting scheme. After spotting of different dilutions of the bacteria, the inoculated agar plates were incubated at 37° C. under anaerobic conditions for 72 h. The growth of the probiotic strains on the agar plates with the different C-sources was evaluated by photo documentation after 24 h, 48 h and 72 h.

### Results

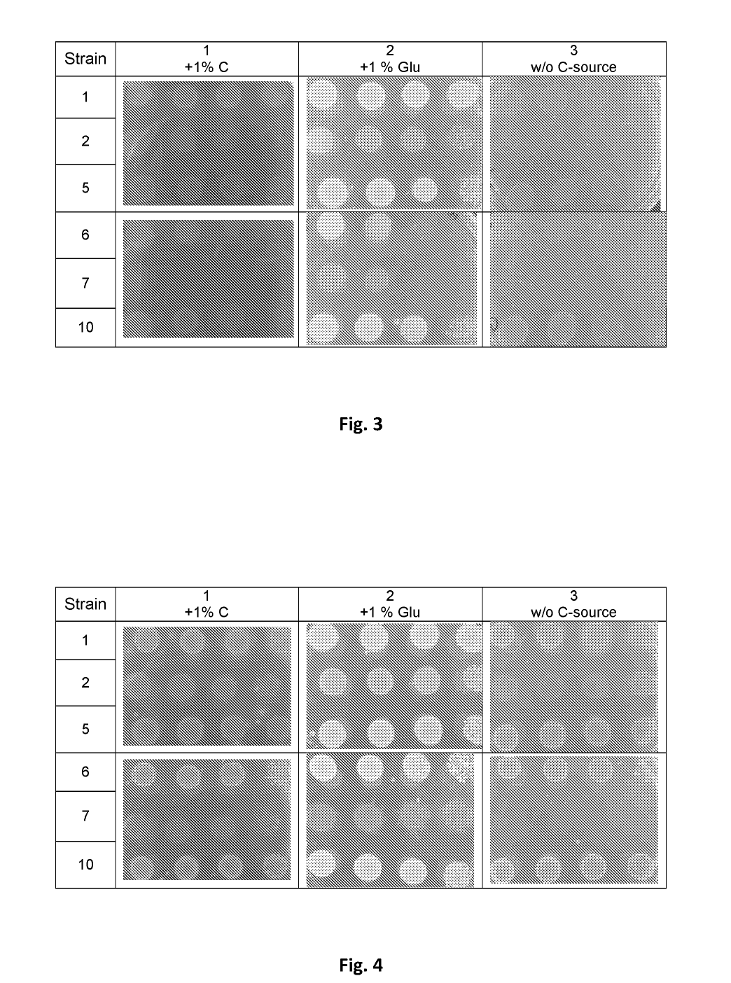

The growth of the probiotic cultures on agar plates was monitored during 72 h of cultivation. The differences in the growth between the strains were most obvious after 24 h and 48 h of cultivation. Therefore these two time points were considered for the evaluation of the strains growth behavior. The results after 24 h of cultivation are shown in FIGS. 2 to 3.

FIG. 2 first of all shows that all strains grew well on glucose as C-source (2). On agar plates without glucose (3) the strains 1, 2, 5, 6 and 10 were able to grow, but to a limited extend compared to the positive control with addition of glucose. Strain 7 however did not grow without a C-source.

On agar plates supplemented with Perilla extract (1) all strains exhibited a growth comparable to growth on agar plates supplemented with glucose. It is therefore concluded that the Perilla extract (plant) can be used as C-source by all strains tested.

The results after 24 h of cultivation of probiotic Lactobacilli strains on LMM medium are shown in FIG. 3.

FIG. 3 first of all shows that all strains grew well on LMM agar plates supplemented with glucose (2; positive control). Without the addition of a C-source all strains showed only a very slight growth (3).

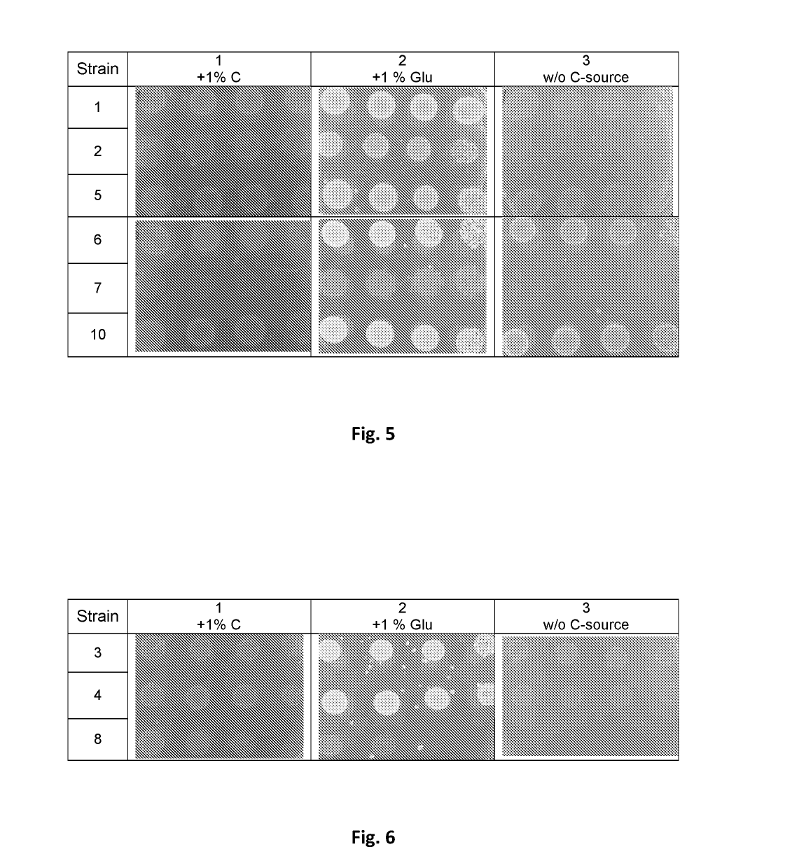

The results of cultivation of probiotic Lactobacilli strains on sMRS and LMM medium after 48 h of incubation are shown in FIGS. 4 and 5. In comparison to 24 h of incubation the growth of the strains on the plant extract formulation after 48 h cultivation is more clear. The strains 1, 5, 6 and 10 showed distinct growth on all plant extract formulations as well as on the negative control plate without a C-source. However, the growth of this strain 1 was significantly better on the Perilla extract (1) compared to the negative control. Strains 2 (Lactobacillus acidophilus) and 7 (Lactobacillus bulgaricus) showed growth on the Perilla extract (1), but not on the negative control plate without a C-source indicating that the plant extract is exclusively used as a carbon source by these two strains.

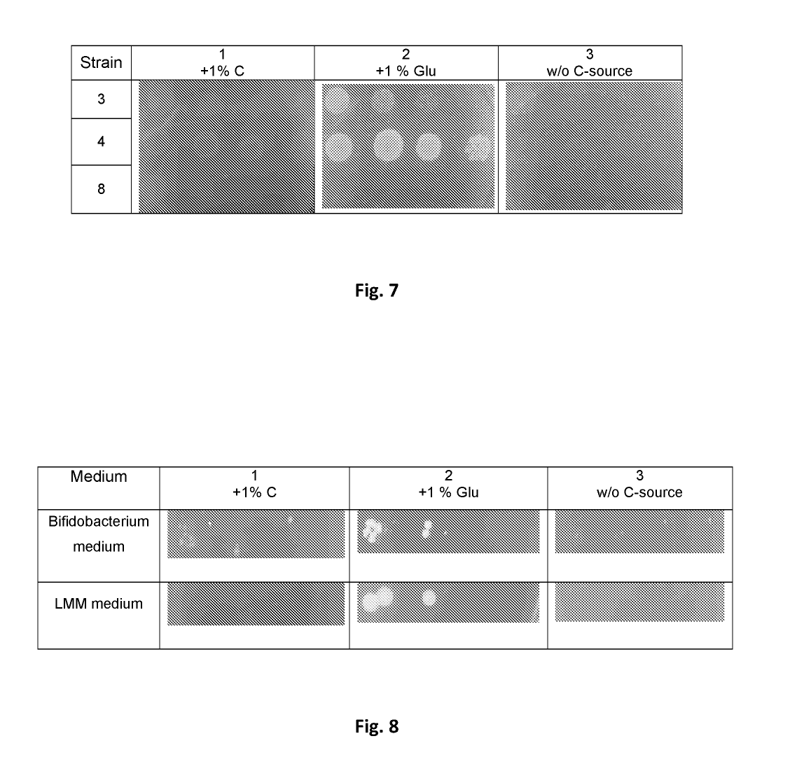

The most significant results for the Bifidobacteria strains were obtained after 48 h of cultivation. The results of cultivation of probiotic Bifidobacteria strains on Bifidobacterium medium and LMM medium after 48 h cultivation are shown in FIGS. 6 and 7.

FIG. 6 shows that all strains of Bifidobacteria grew well on glucose as C-source (2). On agar plates without glucose (3) strains 3 and 4 were still able to grow, but showed reduced growth compared to the positive control with glucose. The strain 8 did not grow without supplementation of a C-source.

All Bifidobacteria strains exhibited growth on Perilla extract (1) comparable with the positive control (glucose). The strains 3 and 4 were able to grow Perilla extract formulations (1) as well as on glucose. Also Strain 8 showed growth on formulation C (Perilla plant extract).

The results derived from growth experiments of Bifidobacteria strains on minimal defined LMM medium (FIG. 7) are generally comparable to the growth on complex Bifidobacterium medium.

The strain 9 (Bifidobacterium longum subsp. longum) was separately tested. The FIG. 8 displays the results obtained after growth of strain 9 on special Bifidobacterium medium and LMM medium after 96 h cultivation.

FIG. 8 shows that strain 9 grew on glucose as C-source (2) on plates with special Bifidobacterium medium as well as on the plates with LMM medium. Strain 9 did not grow without addition of a C-source (negative control). However the strain was able to utilize Perilla plant extract (1) as a C-source, although this, was only observed after cultivation on complex medium as the growth on LMM medium was only very limited.

### Example 2

### Perilla Extract Neutralizes Pathogenic Bacteria of Gastrointestinal Tract

Growth experiments with non-probiotic strains on agar plates were carried out by analyzing strains 1-7.

TABLE 6
List of non-probiotic commensal or pathogenic strains tested.

| Strain | Source |
|---|---|
| 1. Salmonella typhimurium | OB strain collection |
| 2. Escherichia coli | OB strain collection |
| 3. Enterobacter cloacae | OB strain collection |
| 4. Klebsiella pneumoniae | OB strain collection |
| 5. Clostridium difficile | OB strain collection |
| 6. Clostridium perfringens | OB strain collection |
| 7. Bacteroides fragilis | OB strain collection |

The growth behaviour of seven pre-selected non-probiotic (commensal or pathogenic) strains was tested. The strains were classified into two groups, four Enterobacteria, which are facultative anaerobic and three obligate anaerobic bacteria (Clostridia and Bacteroides fragilis). For each of the two classified group typical growth media (e.g. recommended by DSMZ strain collection) were selected and supplemented with formulation C and glucose. The negative control without addition of a C-source was also'prepared.

Growth performance tests were conducted on agar plates under anaerobic conditions.

The following cultivation trials were carried out on agar plates for each of the eight strains:
1. 1 cultivations per strain with the Perilla extract as C-source (1%, weight per volume)—formulation C
2. 1 cultivations per strain with glucose as C-source (1%, weight per volume)—positive control 2
3. 1 cultivations per strain without glucose—negative control

The plant extract formulation was not additionally sterilized before using in assay.

Prior to cultivation experiments, the seven non-probiotic strains were classified into two groups according to their growth requirements and cultivations conditions.

The first group consists of members which belong to the family of Enterobacteriaceae. The members of this family share common characteristics such as oxygen tolerance and requirements for certain nutrients. The selected strains belonging to this group are: Salmonella typhimurium, Escherichia coli, Enterobacter cloacae and Klebsiella pneumonia.

The second group comprises the strains Clostridium difficile, Clostridium perfringens and Bacteroides fragilis, which are all obligate anaerobic.

For each group, Enterobacteria and obligate anaerobic strains, one specific complex medium was used to evaluate the bacterial growth on plant extract formulations.

For the first group comprising members of the Enterobacteriaceae CASO medium (typical complex medium for Enterobacteriaceae) was used for growth experiments. The composition of CASO medium is shown in table 7.

TABLE 7
Composition of CASO medium.

| Substance | Manufacturer | Net weight |
|---|---|---|
| Casein peptone | Merck | 17 g/l |
| BBL phytone peptone | BD | 3 g/l |
| papaic digest | | |
| Sodium chloride | Merck | 5 g/l |
| K₂HPO₄ | Merck | 2 g/l |
| D(+)-Glucose | Merck | 20 g/l |
| monohydrate | | (for positive control) |

For the second group comprising Clostridium difficile, Clostridium perfringens and Bacteroides fragilis a complex medium M110 (complex medium for obligate anaerobic bacteria) was chosen. M110 was used in a modified form without saccharides like cellobiose, maltose and starch to exclude potential carbohydrate sources.

The composition of the M110 medium is displayed in table 8.

TABLE 8
Composition of M110 medium.

| Substance | Manufacturer | Net weight | |
|---|---|---|---|
| Meat extract | Merck | 17 | g/l |
| Casitone | Merck | 30 | g/l |
| Yeast extract | PTU Ohly | 5 | g/l |
| K₂HPO₄ | Merck | 5 | g/l |
| D(+)-Glucose- | Merck | 20 g/l | |
| monohydrate | | (for positive control) | |
| Resazurine | Sigma | 25 | mg/l |
| L-Cystein | Merck | 0.5 | g/l |

### Establishment of Experimental Set-Up and Preparation of Media

For the preparation of the cultivation media 20% (w/v) solutions (in water) of glucose and plant extract formulations were prepared. The plant extract formulation (C) was applied as non sterilized solution. The 20% (w/v) solution of glucose was treated by sterilization.

The agar bases were supplemented with different C-sources just before pouring the media into the petri dishes. The applied concentrations of the plant extract formulation in each agar were 1% (w/v). One positive control was prepared with addition of 1% glucose. As a negative control a medium without addition of a C-source was prepared. In total three preparations per medium were used.

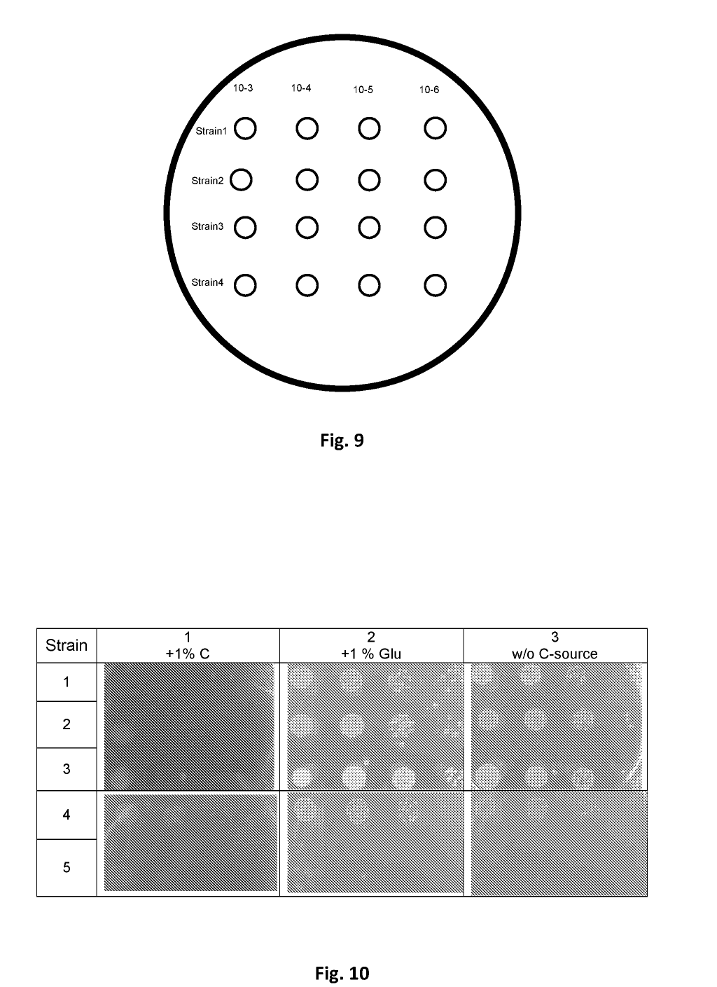

After the preparation of agar plates, different dilutions of each of the non-probiotic strains (10⁻³ to 10⁻⁶) after pre-cultivation and subsequent washing with PBS were prepared and spotted onto the agar plates. FIG. 9 shows an example for a corresponding spotting scheme.

The inoculated agar plates were incubated at 37° C. under anaerobic conditions for several days. The growth of the non-probiotic strains on the agar plates with the different C-sources was visually evaluated by photo documentation in suitable time intervals dependent on the strain.

### Results

The most adequate time point for the differentiation of growth of each of the strains was chosen, because the growth of the strains was very diverse.

### Enterobacteria

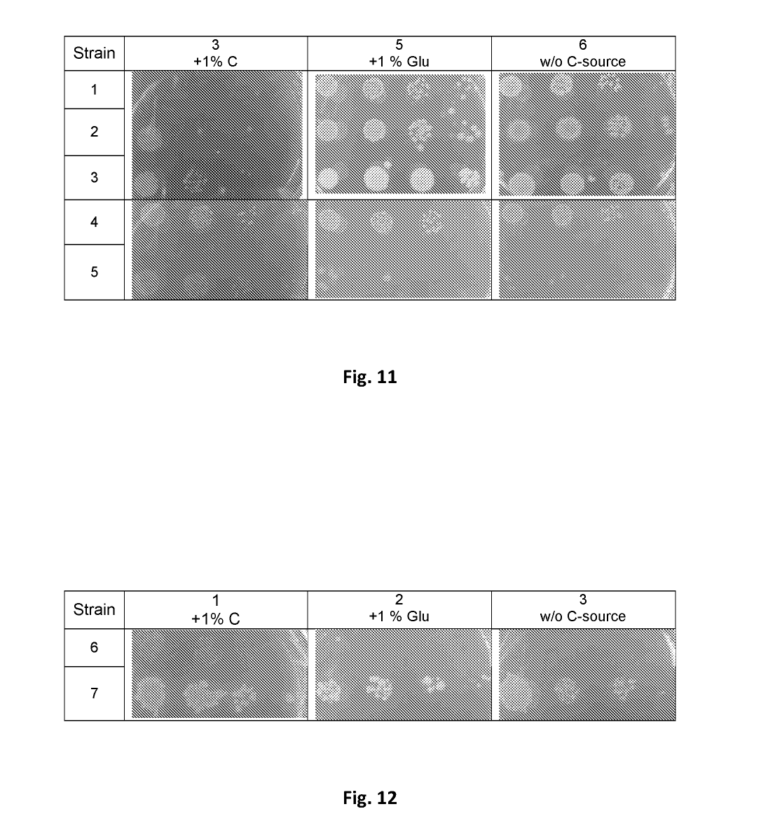

The Enterobacteria are characterized by rapid growth on complex media. So, for the cultivation experiments of these strains high dilutions of cells were prepared. The first evaluation of growth occurred after 16 h of incubation. The results of growth experiment on CASO medium after 16 h and 24 h of cultivation are shown in FIGS. 10 and 11.

The results shown in FIGS. 10 and 11 exhibit that the strains 1, 2, 3 and 4 grow very well on CASO medium containing glucose as C-source. On agar plates without the addition of a C-source (3; negative control) these strains exhibited a growth similar to the growth on glucose.

The fact that the strains exhibited growth on CASO medium without the addition of a C-source (3) indicates that the cells were able to metabolize other nutrients in the medium as a C-source.

However the growth of pathogenic strains 2 and 3 was delayed by Perilla extract formulation (1). It was characterized by significantly limited growth of dilution steps of strain 2 (Escherichia coli) and strain 3 (Enterobacter cloacae). The growth of strain 1 (Salmonella typhimurium) was completely inhibited by Perilla extract formulation. The strain was not able to use the plant extract as an energy source.

In case of strain 4 (Klebsiella pneumoniae) no significant differences were found between different C-sources. This strain was able to grow on the tested plant extract formulation (1) as well as on glucose. These results demonstrate that plant extract formulations have no effect on the growth of K. pneumoniae on CASO medium.

### Obligate Anaerobic Bacteria

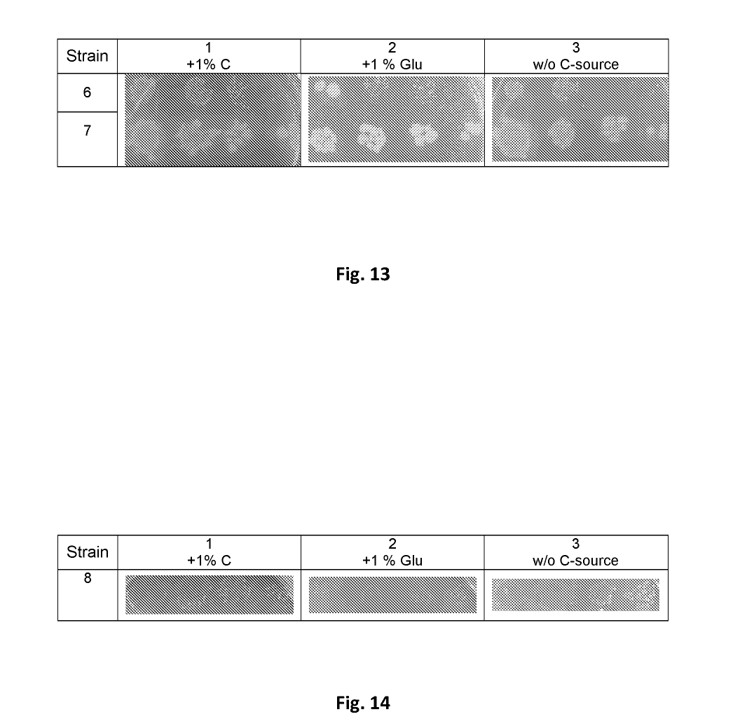

Results of the growth experiments of two Clostridia strains 5 (Clostridium difficile) and 6 (Clostridium perfringens) on M110 medium after 24 h cultivation are shown in FIG. 12. FIG. 13 illustrates the results after 48 h of cultivation.

The FIGS. 12 and 13 indicate that C. difficile (strain 5) and C. perfringens (strain 6) are able to metabolize Perilla extract formulation (1) as a C-source.

These strains exhibit a reduced growth on the control media with the addition of glucose at a concentration of 2%.

Strain 7 (Bacteroides fragilis) exhibits slow growth on the agar plates. Therefore the most suitable results could be monitored after 7 days of cultivation of this strain. FIG. 14 displays the growth of strain 7 on different C-sources.

The results of the cultivation experiments with Bacteroides fragilis (FIG. 14) demonstrate that the different C-sources had no influence on the growth of this strain. When the plant extract formulation was applied as a C-source the growth of B. fragilis was also not stimulated or reduced. Therefore these results demonstrate that the plant extract formulation have no effect on the growth B. fragilis in M110 medium.

### Summary of the Examples

Ten probiotic strains and seven non-probiotic strains were tested on their ability to utilize Perilla plant extract formulation as a C-source. For that purpose growth experiments on agar plates supplemented either with glucose (positive control) (2), Perilla plant extract formulation (1) or without a C-source (negative control) (3) were performed. Three different basic media compositions were used for growth experiments.

The growth experiments with probiotic Lactobacilli and Bifidobacteria demonstrated that all tested probiotic strains are able to grow on media supplemented with Perilla extract (C) as C-source. All these strains showed a growth which is equal to the growth on glucose as C-source. This is remarkable as glucose is usually considered as the optimal C-source. Three strains (strain 7—Lactobacillus bulgaricus, strain 8—Bifidobacterium bifidum and strain 9 Bifidobacterium longum) showed no growth on the negative control agar plates (both basic media sMRS and LMM without C-source). Strain 2 (Lactobacillus acidophilus) also showed no growth on the minimal LMM medium without a C-source but very slight growth on complex medium without C-source.

This means that these four strains exclusively used Perilla extract as the only C-source on the corresponding plates. These four strains would be very suitable for the development of a synbiotic Perilla extract (prebiotic) in combination with one these strains.

Another six strains, which grew on complex media without a C-source, seem to be able to use components of the complex ingredients (yeast extract, peptone) of the medium base (sMRS or Bifidobacterium medium) as a C-source for growth. Hence, it cannot be excluded that the growth of these six strains on plant extract formulations supplemented media is a combinatory effect of this compound in complex media base and plant extract formulation.

The results of the growth assays show no significant differences between complex and defined minimal medium, although in some cases the results obtained from cultivation on minimal media were more distinct comparable to the complex medium because of low back ground growth.

The first growth experiments with non-probiotic strains on CASO medium revealed that four of the tested Enterobacteria strains were able to grow well on glucose as C-source. On medium without the addition of a C-source (3; negative control) these four strains exhibited a growth similar to the growth on glucose. As CASO is a complex medium rich in nutrients, some of compounds could be used as a C-source for growth of bacteria.

The growth of the strains 4 and 5 was not stimulated by the plant extract, which complies well with a prebiotic substance.

The growth of pathogenic strains 2 (Escherichia coli) and 3 (Enterobacter cloacae) was even inhibited (significantly delayed) by the Perilla extract formulation (C). The growth of strain 1 (Salmonella typhimurium) was completely inhibited by native Perilla extract formulation.

The inhibition of these three strains by the Perilla extract is remarkable as not only a neutral effect on the pathogenic bacteria was observed, but a detrimental effect. This would strongly influence the prebiotic index in a positive way.

No significant differences in growth of Klebsiella pneumoniae (strain 4) on different C-sources were observed. This strain was able to grow on the tested plant extract formulation (1) as well as on glucose. These results demonstrate that Perilla extract formulation has no effect on the growth of K. pneumoniae on CASO medium agar plates under the tested conditions, which complies well with a prebiotic substance.

The growth of obligate anaerobic strains was tested on M110 complex medium. The results demonstrate that the growth of none of the strains was enhanced when the Perilla extract formulations were added to the media.

Summarizing, the inventors surprisingly found that Perilla preparation has prebiotic activity. This is an advantageous finding since Perilla preparation can be administered at a low dosage compared to prior art prebiotics. In addition, Perilla does not cause any side effects, the prebiotic effect starts within 24 h at the administration and the effect is stable in the gastrointestinal tract. Thus, Perilla extract may be used for various gastrointestinal indications as well as for indications for which prebiotic agents are useful in other parts of the human or animal body.

### LITERATURE

1. Sekirov et al. 2010 Gut Microbiota in health and disease. Physiol Rev 90: 859-904.
2. Schrezenmeier and de Vrese 2001 Probiotics, prebiotics, and synbiotics—approaching a definition. Am J Clin Nutr 73(suppl):361-364.
3. Roberfroid et al. 2010 Prebiotic effects: metabolic and health benefits. Brit J of Nutr 104: 007-1145.
4. World Gastroenterology Organisation Practice Guideline 2008 Probiotics and prebiotics.
5. Collins and Gibson 1999 Probiotics, prebiotics, and synbiotics: approaches for modulating the microbil ecology of the gut. Am J Clin Nutr 69(suppl):1052(S)-7S.
6. Mandalari et al. 2007 In vitro evaluation of the prebiotic activity of a pectic oligosaccharide-rich extract enzymatically derived from bergamot peel. Appl Microbiol Biotechnol 73:1173-9.
7. Manderson et al. 2005 In vitro determination of prebiotic properties of oligosaccharides derived from an orange juice manufacturing by-product stream. Appl Environ Microbiol 71:8383-9.
8. Britton and Versalovic 2008 Probiotics and Gastrointestinal Infections. Interdiscip Perspect Infect Dis., Vol. 2008.
9. Iqbal et al. 2014 Probiotics and their beneficial effects against various diseases. Pak. J. Pharm. Sci., Vol. 27, No. 2: 405/15.
10. Roberfroid 2007 Prebiotics: The Concept Revisited. J. Nutr. 137: 830-7.

1. A method of promoting growth of probiotic bacteria in a subject and/or inhibiting the growth of pathogenic bacteria selected from the group consisting of Enterobacteriaceae, Bacteroides and/or Clostridia in a subject, the method comprising administering to the subject a prebiotic composition comprising a Perilla preparation in an amount effective to promoting growth of probiotic bacteria in a subject as a prebiotic and/or inhibiting the growth of pathogenic bacteria selected from the group consisting of Enterobacteriaceae, Bacteroides and/or Clostridia.

2. The method according to claim 1, wherein the preparation is a Perilla extract.

3. The method according to claim 1, wherein the preparation is a Perilla frutescens or Perilla ocymoides preparation.

4. The method according to claim 1, wherein the probiotic bacteria is selected from the group consisting of Bifidobacteria and Lactobacilli.

5. The method according to claim 4, wherein the probiotic bacteria is selected from the group consisting of Lactobacillus bulcaricus, Lactobacillus plantarum, Lactobacillus acidophilus, Lactobacillus fermentum, Lactobacillus casei, Lactobacillus rhamnosus, Bifidobacterium bifidum, Bifidobacterium lactis and Bifidobacterium longum.

6. The method according to claim 1 wherein the Perilla preparation has a neutral effect to the growth of pathogenic bacteria.

7. The method according to claim 1, wherein the Perilla preparation neutralizes pathogenic bacteria.

8. The method according to claim 7 wherein the Perilla preparation inhibits the growth of pathogenic bacteria.

9. The method according to claim 1, wherein the pathogenic bacteria are selected from the group consisting of Enterobacteriaceae, Bacteroides and Clostridia.

10. The method according to claim 9, wherein the pathogenic bacteria are selected from the group consisting of Salmonella typhimurium, Escherichia coli and Enterobacter cloacae.

11. The method according to claim 1, wherein the composition further comprises a further prebiotic agent, a fiber, a probiotic agent, a lipid, a physiologically active fatty acid, a sterol, a sterol ester, a stanol ester, a bulking agent, a medicament, an antispasmodic or an anti-inflammatory agent, an antioxidative agent, plant phenolic, a phenolic metabolite, a catechin, a vitamin, a mineral, essential oil or a plant preparation.

12. The method according to claim 1, wherein the concentration of the Perilla preparation from fresh leaves is in the range of 2 mg to 400 g, from dried leaves is in the range of 0.25 mg to 50 g or from Perilla extract or tea is in the range of 0.05 mg to 10 g.

13. The method according to claim 1, wherein the composition is comprised in a food product, dietary supplement, medical device or medicament.

14. The method according to claim 1, wherein administering the composition maintains digestive health, maintains healthy gut microflora, maintains a normal digestion, improves gut regularity, supports healthy gut mobility, bowel movement and/or healthy stool frequency, stool consistency and/or form, improves the immune system, skin and/or hair, regulates the lipid metabolism, improves glucose homeostasis, weight management and/or the mouth flora or improves bowel function.

15. The method according to claim 1, wherein administering the composition reduces bloating, reduces distension, reduces passage of gas, reduces stomach rumbling, reduces feeling of fullness, ameliorates constipation, ameliorates diarrhea, reduces visceral hypersensitivity, reduces abdominal discomfort like cramps or pain, preventing prevents or reduces inflammatory bowel disease or irritable bowel syndrome (IBS) and/or colon cancer, reduces and/or inhibits growth of pathogenic bacteria, treats or prevents gut infections, treats allergies or immune diseases, prevents and/or reduces atopic dermatitis, prevents caries, prevents skin and/or hair disorders or lowers cholesterol and/or triacylglycerol plasma concentrations.

16. The method according to claim 15, wherein the gut infection is travel diarrhea or food infection.

17. (canceled)

18. (canceled)

19. (canceled)

20. (canceled)

21. A method of treating or preventing gut infections in a subject, the method comprising administering to the subject a prebiotic composition comprising a Perilla preparation in an amount effective to treat or prevent gut infections in the subject.

22. The method of claim 21, wherein the gut infection is travel diarrhea or food infection.

23. A method of promoting growth of probiotic bacteria in vitro, the method comprising incubating probiotic bacteria with a prebiotic composition comprising a Perilla preparation in an amount effective to promote growth of probiotic bacteria in vitro.

24. A method of inhibiting the growth of pathogenic bacteria in vitro, the method comprising incubating pathogenic bacteria with a prebiotic composition comprising a Perilla preparation in an amount effective to inhibit the growth of pathogenic bacteria in vitro, wherein the pathogenic bacteria is selected from the group consisting of Enterobacteriaceae, Bacteroides and/or Clostridia.

25. The method according to claim 2, wherein the preparation is a liquid, tea or powder.

26. The method according to claim 3, wherein the preparation is a preparation of aerial parts of Perilla.

27. The method according to claim 26, wherein the preparation is a leaf or stem preparation.
